(19) United States
(12) Patent Application Publication
(10) Pub. No.: US 2021/0322705 A1
(43) Pub. Date: Oct. 21, 2021

(54) Face Covering Apparatus

(71) Applicant: Erick Koji Hasegawa, Redwood City, CA (US)

(72) Inventor: Erick Koji Hasegawa, Redwood City, CA (US)

(21) Appl. No.: 16/852,460

(22) Filed: Apr. 18, 2020

(51) Int. Cl. A61M 16/06; A41D 13/11

(52) U.S. Cl. CPC A61M 16/0688; A61M 16/0605; A61M 2202/206; A61M 2205/02; A41D 13/1169

(57) ABSTRACT

An embodiment may include a face covering apparatus. The face covering apparatus includes one or more machine-washable layers of cloth, where a plurality of magnets is insertable in and removable from the one or more machine-washable layers of cloth. The face covering apparatus includes a plurality of adhesive magnets comprising a plurality of dual-sided adhesives, where the plurality of dual-sided adhesives is configured to secure the plurality of adhesive magnets to a human face. The plurality of cloth magnets is configured to secure the one or more machine-washable layers of cloth over the human face based at least on a magnetic attraction between the plurality of cloth magnets inserted in the one or more machine-washable layers of cloth and the plurality of adhesive magnets secured to the human face.

### BACKGROUND

A conventional face mask may be used to block various types of microorganisms, agents, and other particles not visible by the human eye. For example, a face mask may be used to block harmful agents, such as various types of viruses. As the number of viruses increase in various societies around the world, particularly novel viruses, a face mask may be imperative to ensure safety and health, particularly based on a person's age or preexisting health condition. In addition, and considering circumstances holistically, the face mask may be imperative to ensure the health of those around that person. Therefore, face masks may be critical to ensure the health of ourselves and others around us.

### SUMMARY

One of the many challenges with conventional face coverings and face masks with ear loops, elastic bands, and ties is that they require constant adjustments throughout the duration of use. For example, the use of elastic bands and ties around the ears is uncomfortable and often painful, particularly if used for longer periods of times (e.g., long shifts at the hospital, long wait times at the grocery store or the pharmacy due to social distancing restrictions, etc.) In addition, the discomfort and pain causes the wearer to periodically make adjustments to the ear loops, elastic bands, and ties, thereby transferring harmful agents from their hands to the loops, bands, and ties, and vice versa. Thus, these agents may be transferred to the ears, the eyes, and/or mouth, potentially exacerbating the problem with the rapid spread of harmful agents. It is desirable to have a face covering that solves these problems.

Accordingly, a first example embodiment may be a face covering apparatus including one or more machine-washable layers of cloth, where a plurality of magnets is insertable in and removable from the one or more machine-washable layers of cloth. A plurality of adhesive magnets may include a plurality of dual-sided adhesives. The plurality of dual-sided adhesives is configured to secure the plurality of adhesive magnets to a human face. The plurality of cloth magnets may be configured to secure the one or more machine-washable layers of cloth over the human face based at least on a magnetic force or attraction between the plurality of cloth magnets inserted in the one or more machine-washable layers of cloth and the plurality of adhesive magnets secured to the human face.

The first example embodiment may involve the one or more machine-washable layers of cloth covering a nose and a mouth of the human face of a human person without the one or more machine-washable layers of cloth making contact with one or more ears of the person, a neck of the human person, a back of a head of the human person, and a top of the head of the human person.

The first example embodiment may involve a plurality of dual-sided adhesives that is removable from the plurality of adhesive magnets and replaceable with a second plurality of dual-sided adhesives configured to secure the plurality of adhesive magnets to the human face.

The first example embodiment may involve the plurality of cloth magnets inserted between a first layer and a second layer of the one or more machine-washable layers of cloth, where the plurality of cloth magnets secure the one or more machine-washable layers of cloth over the human face based at least on the magnetic force or attraction through the second layer to the plurality of adhesive magnets secured to the human face.

The first example embodiment may involve the one or more machine-washable layers of cloth including a plurality of pockets and a plurality of insertion slots, where the plurality of cloth magnets is insertable in the plurality of insertion slots to secure the plurality of cloth magnets. The plurality of cloth magnets secure the one or more machine-washable layers of cloth over the human face based at least on the magnetic force or attraction through the plurality of insertion slots to the plurality of adhesive magnets secured to the human face.

The first example embodiment may involve a lightweight metallic wire that is insertable in and removable from the one or more machine-washable layers of cloth. The lightweight metallic wire is bendable and flexible to meet one or more contours of the human face. The lightweight metallic wire is configured to secure the one or more machine-washable layers of cloth over the human face based at least on a magnetic attraction between the lightweight metallic wire inserted in the one or more machine-washable layers of cloth and the plurality of adhesive magnets secured to the human face.

The first example embodiment may involve the plurality of adhesive magnets configured to attach to a pair of eyeglasses. At least one of the plurality of cloth magnets is configured to secure the one or more machine-washable layers of cloth over the human face based at least on a magnetic attraction between at least one of the plurality of cloth magnets in the one or more machine-washable layers of cloth and at least one of the plurality of adhesive magnets secured to the pair of eyeglasses. The one or more machine-washable layers of cloth covers a nose and a mouth of the human face of a human person without the one or more machine-washable layers of cloth making contact with one or more ears of the human person, a neck of the human person, a back of a head of the human person, and a top of the head of the human person.

The first example embodiment may involve at least one of the plurality of adhesive magnets that is adjustable to attach to a bridge of a pair of eyeglasses. The one or more machine-washable layers of cloth includes at least one pocket and at least one insertion slot. At least one of the plurality of cloth magnets is insertable in at least one insertion slot to secure at least one of the plurality of cloth magnets. At least one of the plurality of cloth magnets secures the one or more machine-washable layers of cloth over the human face based on the magnetic force or attraction through the insertion slot to the plurality of adhesive magnets attached to the bridge of the pair of eyeglasses.

The first example embodiment involves the plurality of adhesive magnets configured to attach to a pair of wireless headphones. The plurality of cloth magnets is configured to secure the one or more machine-washable layers of cloth over the human face based at least on a magnetic attraction between the plurality of cloth magnets in the one or more machine-washable layers of cloth and the plurality of adhesive magnets secured to the pair of wireless headphones. The one or more machine-washable layers of cloth covers a nose and a mouth of the human face of a human person without the one or more machine-washable layers of cloth making contact with a neck of the human person, a back of a head of the human person, and a top of the head of the human person.

The first example embodiment involves the plurality of adhesive magnets adjustable to attach to the pair of wireless headphones. The one or more machine-washable layers of cloth includes a plurality of pockets and a plurality of insertion slots. The plurality of cloth magnets is insertable in the plurality of insertion slots to secure the plurality of cloth magnets with the one or more machine-washable layers of cloth. The plurality of cloth magnets secures the one or more machine-washable layers of cloth over the human face based on the magnetic attraction through the plurality of insertion slots to the plurality of adhesive magnets attached to the pair of wireless headphones.

The first example embodiment involves the plurality of adhesive magnets attached to a pair of eyeglasses. One or more other adhesive magnets from the plurality of adhesive magnets is attached to a pair of wireless headphones. The plurality of cloth magnets is configured to secure the one or more machine-washable layers of cloth over the human face based at least on one or more magnetic attractions between the plurality of cloth magnets in the one or more machine-washable layers of cloth and at least one of the plurality of adhesive magnets attached to the pair of eyeglasses and the one or more other adhesive magnets attached to the pair of wireless headphones.

The first example embodiment involves a first face covering including a single layer of cloth, a second face covering including a double layer of cloth and a third face covering including a triple layer of cloth. The single layer of cloth is removable from the plurality of adhesive magnets secured to the human face and replaceable with either the second face covering or the third face covering without having to remove the plurality of adhesive magnets secured to the human face. The single layer of cloth is removable and replaceable without making contact with one or more ears of the person, a neck of the human person, a back of a head of the human person, and a top of the head of the human person.

The first example embodiment involves the plurality of dual-sided adhesives including at least one of the following organic adhesives: starch adhesives, paste-based adhesives, natural resin adhesives, and animal adhesives.

The second example embodiment involves a face mask apparatus including one or more layers of cloth. The face mask includes a plurality of magnets insertable in and removable from the one or more layers of cloth. The face mask also includes a plurality of adhesive magnets comprising a plurality of dual-sided adhesives, where the plurality of dual-sided adhesives is configured to secure the plurality of adhesive magnets to a human face. The plurality of cloth magnets is configured to secure the one or more layers of cloth over the human face based at least on a magnetic attraction between the plurality of cloth magnets inserted in the one or more layers of cloth and the plurality of adhesive magnets secured to the human face.

The second example embodiment also involves the face mask taking the form of a surgical mask and/or a respirator mask. The first face mask is a surgical mask and a second face mask is a respirator mask. The surgical mask is removable from the plurality of adhesive magnets secured to the human face and replaceable with the respirator mask without removing the plurality of adhesive magnets secured to the human face of a human person. The surgical mask is removable and replaceable without making contact with one or more ears of the human person, a neck of the human person, a back of a head of the human person, and a top of the head of the human person.

The second example embodiment involves a lightweight metallic wire insertable in and removable from the one or more layers of cloth. The lightweight metallic wire is bendable to meet one or more contours of the human face. The lightweight metallic wire is configured to secure the one or more layers of cloth over the human face based at least on a magnetic attraction between the lightweight metallic wire inserted in the one or more layers of cloth and the plurality of adhesive magnets secured to the human face.

The third example embodiment, involves a method for providing a face covering apparatus. The first step is for providing one or more machine-washable layers of cloth such that a plurality of magnets is insertable in and removable from the one or more machine-washable layers of cloth. The second step is for providing a plurality of adhesive magnets including a plurality of dual-sided adhesives. The plurality of dual-sided adhesives is configured to secure the plurality of adhesive magnets to a human face. The plurality of cloth magnets is configured to secure the one or more machine-washable layers of cloth over the human face based at least on a magnetic attraction between the plurality of cloth magnets inserted in the one or more machine-washable layers of cloth and the plurality of adhesive magnets secured to the human face.

These as well as other embodiments, aspects, advantages, and alternatives will become apparent to those ordinary skill in the art by reading the following detailed description with reference where appropriate to accompanying drawings. Further, this summary and other descriptions and figures provided herein are intended to illustrate embodiments by way of example only, and as such, that numerous variations are possible. For example, structure elements and process steps can be rearranged, combined, distributed, eliminated, or otherwise changed while remaining within the scope of the embodiments claimed.

### BRIEF DESCRIPTION OF THE DRAWINGS

FIG. 1 illustrates a drawing of a face covering with ties, in accordance with example embodiments.

FIG. 2A illustrates a drawing of a face covering, in accordance with example embodiments.

FIG. 2B illustrates a drawing of a face covering from a frontal view, in accordance with example embodiments.

FIG. 2C illustrates a drawing of an exploded view of the face covering, in accordance with example embodiments.

FIG. 2D illustrates a drawing of an inside view of the face covering, in accordance with example embodiments.

FIG. 3A illustrates a drawing of a face covering in relation to a pair of glasses, in accordance with example embodiments.

FIG. 3B illustrates a drawing of a face covering in relation to a pair of glasses from a frontal view, in accordance with example embodiments.

FIG. 3C illustrates a drawing of an exploded view of the face covering in relation to a pair of glasses, in accordance with example embodiments.

FIG. 3D illustrates a drawing of an inside view of the face covering, in accordance with example embodiments.

FIG. 4A illustrates a drawing of a face covering in relation to a pair of wireless headphones, in accordance with example embodiments.

FIG. 4B illustrates a drawing of a frontal view of a face covering in relation to a pair of wireless headphones, in accordance with example embodiments.

FIG. 4C illustrates a drawing of an exploded view of a face covering in relation to a pair of wireless headphones, in accordance with example embodiments.

FIG. 4D illustrates a drawing of an inside view of a face covering in relation to a pair of wireless headphones, in accordance with example embodiments.

FIG. 5 illustrates a flowchart, in accordance with example embodiments.

### DETAILED DESCRIPTION

Very recently, the Centers for Disease Control and Prevention (CDC) released recommendations to wear cloth face coverings to help prevent the spread of infectious agents, particularly the novel coronavirus also known as COVID-19. The recommendations urge people to wear cloth face covering in public settings. In some cases, it may be challenging to maintain social distancing measures in public settings. For example, it may be difficult to maintain six feet of distance between other people in narrow grocery aisles and pharmacies, among other establishments that may be essential to ensure the health and well-being of people under shelter-in-place restrictions. Thus, the recommendations indicate that these face coverings should be used especially in areas of significant community-based transmission.

The recommendations further advise the use of simple cloth face coverings. The recommendations described above also note that the cloth face covering should not be surgical masks or N-95 respirators. It is noted that these masks and respirators are imperative supplies that must be reserved to help protect our healthcare workers and first responders working tirelessly to help those affected by the virus, among other harmful agents. Thus, as an additional public measure, the recommendations indicate that the simple cloth face coverings will help stop people from spreading the virus, particularly those who may be asymptomatic and thus, they may not know they carry the virus. As such, the advice is to use these cloth face coverings in public settings, where the coverings may be created from scratch and particularly from household items and common materials available at low cost.

The recommendation also indicates the cloth face coverings should be secured with ties, elastic bands, or ear loops. As noted above, one of the many challenges with these is that they require constant adjustments throughout the duration of using these cloth face coverings. For example, the recommendation describes steps in creating a sewn cloth face covering with elastic bands, ear loop, hair ties, or headbands. The use of these items around the ears is quite uncomfortable and often painful, particularly if used for longer periods of times (e.g., long wait times at the grocery store or the pharmacy due to social distancing restrictions). In addition, the discomfort and pain causes the wearer to periodically make adjustments to these items, thereby transferring harmful agents from their hands to these bands, loops, and ties, and vice versa. Thus, these agents may be transferred to the ears and various locations around the eyes and/or mouth, potentially exacerbating the problem with the rapid spread of harmful agents.

In another example, the recommendations describe steps in creating a face covering using a t-shirt. The covering involves narrow strips of the t-shirt tied around the top of the head and tied around the neck. One of the many challenges with these ties is that they begin to loosen over time as the wearer moves. And similar to the ties and ear loops described above, the ties around the head and neck have to be adjusted and often re-tied to keep the cloth covering around the nose and mouth. Thus, harmful agents may be transferred from the wearer's hands to the ties around the head and neck, and vice versa.

As such, the exemplary embodiments described herein resolve these problems. For example, the face covering described herein may include one or more machine-washable layers of cloth. Further, the one or more machine-washable layers of cloth may be configured and/or adapted to fit closely and comfortably over a human person, possibly resting against the side of the face. The one or more machine-washable layers of cloth, including various types of fabric, may be configured and/or adapted to allow for breathing without restricting the breathing. The one or more machine-washable layers of cloth may also be durable such that the layers of cloth may be laundered and machine dried on a daily basis without damage or change in the shape of the layers of cloth. The one or more machine-washable layers of cloth may include layers of cotton, fine knitted fabrics made of natural or man-made fibers, synthetic fibers for elasticity, microfibers, satin, foam for form, mesh, lace, among other types of cloth layers and fabric, and potentially combinations of these to allow for breathing without restricting the breathing.

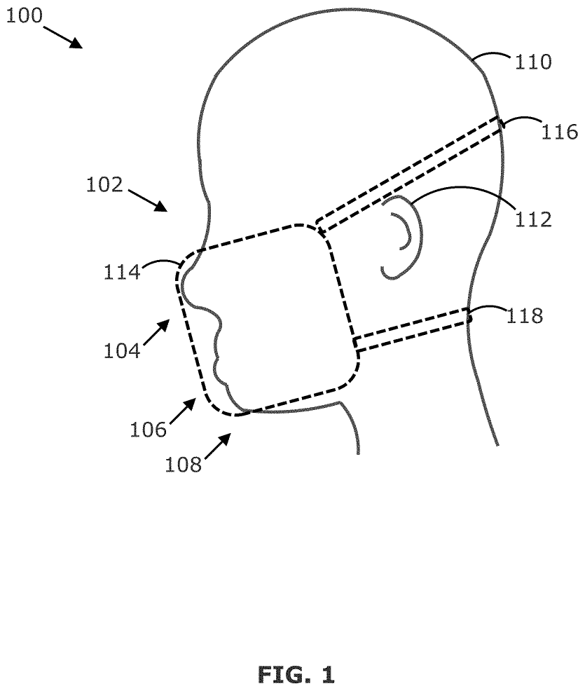

As noted above, FIG. 1 illustrates a drawing of a face mask 114 with ties 116 and 118, in accordance with example embodiments. As shown, human person 100 includes human face 102. Human face 102 may include nose 104, mouth 106, and chin and/or neck 108. Further, human person 100 includes head 110, ears 112, among various other parts of the body of human person 100. As shown, face mask 114 includes upper tie 116 that wraps around head 110 and lower tie 118 that wraps around neck 108.

As noted above, one of the challenges with ties 116 and 118 is they loosen over time as the human person 100 moves, particularly with movement around chin and/or neck 108. Other examples described above include ties 116 and 119 wrapping around ear 112 to secure face mask 114 to human face 102. Similarly, in such examples, ties 116 around head 110 and neck 108 have to be adjusted and often re-tied to keep face mask 114 around nose 114 and mouth 106. As described above, human person 100 may periodically make adjustments to ties 116 and 118, thereby transferring harmful agents from their hands to ties 116 and 118, and vice versa. As such, these harmful agents may also transfer to ear 112, neck 108, head 110, among other areas of human face 100. In some circumstances, harmful agents may be transferred to nose 104, mouth 106, and/or eyes of human person 100, thereby increasing chances for human person 100 getting infected with harmful agents.

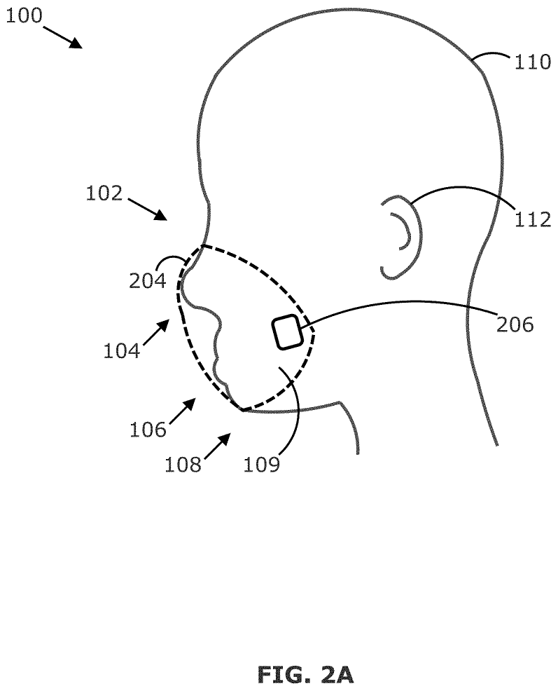

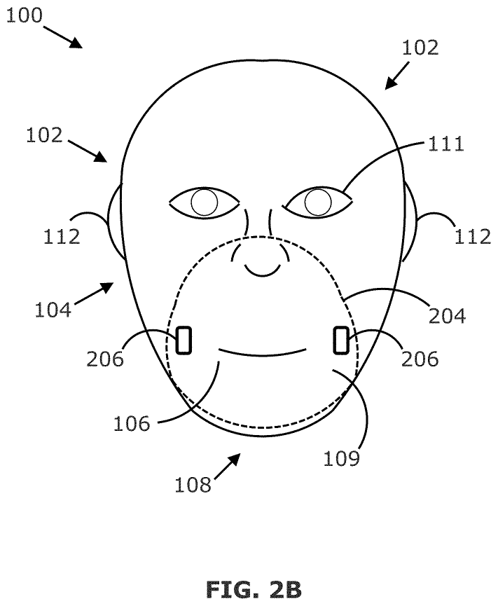

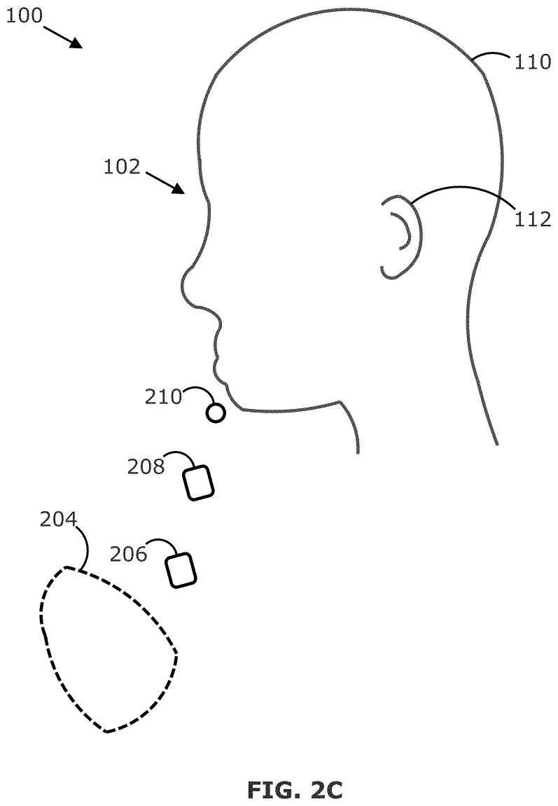

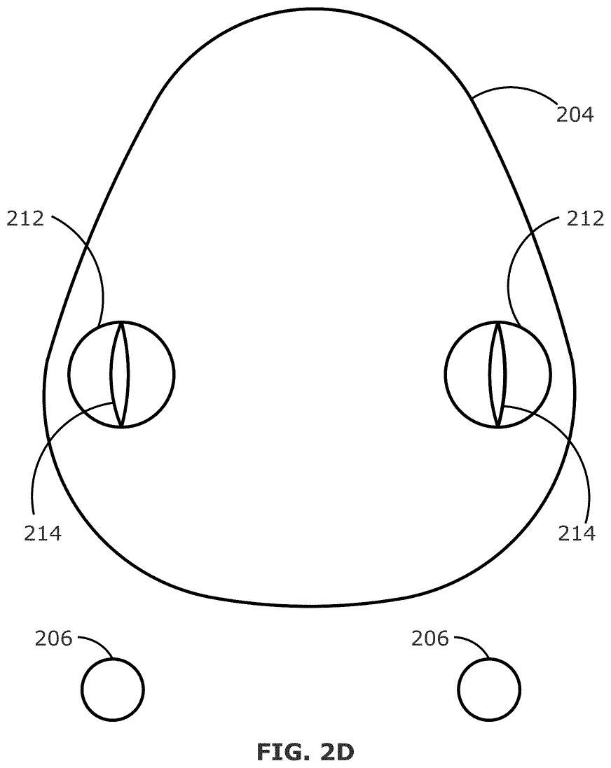

FIG. 2A illustrates a drawing of a face covering 204, in accordance with example embodiments. As shown, human person 100 may include human face 102, nose 104, mouth 106, chin and/or neck 108, facial areas 109 adjacent to nose 104 and mouth 106 (e.g., facial cheeks), head 110, and ears 112, such as those described above. Further, in FIG. 2A, face covering 204 includes a number of magnets 206 insertable in and removable from one or more machine-washable layers of cloth of face covering 204. For example, magnets 206 may be inserted in one or more machine-washable layers of cloth 204 during use and magnets 206 may be removed from one or more machine-washable layers of cloth 204 to be machine-washed and/or machine-dried. Yet, in some embodiments, magnets 206 may be embedded and/or stitched into one or more machine-washable layers of cloth 204. In such instances, magnets 206 may be machine-washable and machine-dryable.

FIG. 2B illustrates a drawing of face covering 204 from a frontal view, in accordance with example embodiments. As shown in FIG. 2A, face covering 204 includes a number of magnets 206 insertable in and removable from one or more machine-washable layers of cloth of face covering 204. A number of magnets 206 may be inserted in one or more machine-washable layers of cloth of face covering 204 such that face covering 204 covers nose 104 and mouth 106 without covering and/or making contact with eyes 111, ears 112, chin and/or neck 108, facial areas 109, and/or head 110, among other portions of human face 102. Since there are no ties, such as ties 116 and 118 described above, the need to make the adjustments above is eliminated, thereby significantly reducing the risks of harmful agents transferring from the hands of human person 100 to ear 112, the back of neck 108, head 110, among other areas of human face 100. Further, by significantly reducing those risks, face covering 204 prevents harmful agents from transferring to nose 104, mouth 106, facial areas 109 adjacent to nose 104 and/or mouth 106 (e.g., facial cheeks), and/or eyes of human person 100, thereby reducing chances for human person 100 getting infected with harmful agents.

FIG. 2C illustrates a drawing of an exploded view of face covering 204, in accordance with example embodiments. In some embodiments, a number of adhesive magnets 208 may include a number of dual-sided adhesives 210, where the number of dual-sided adhesives 210 may be configured to secure, attach, and/or position the number of adhesive magnets 208 to human face 102. Dual-sided adhesives 210 may include natural adhesives that are environmentally-friendly. For example, natural adhesives may be made from organic sources such as vegetable starch (e.g., dextrin), paste-based adhesives (e.g., from cooking flour in water), natural resin adhesives, and/or animals (e.g. the milk protein casein and hide-based animal glues), among other types of bioadhesives. Dual-sided adhesives may include organic adhesives, starch-based adhesives, wallpaper adhesives, casein glue, albumen made from protein components, masonite, organic polymer, synthetic thermosetting resins, and/or pressure sensitive adhesives, among other possibilities.

As shown, the number of dual-sided adhesives 210 may be configured to secure, attach, and/or position the number of adhesive magnets 208 to human face 102. The number of cloth magnets 206 may be configured to secure the one or more machine-washable layers of cloth 204 over human face 102 based at least on a magnetic attraction and/or force between the number of cloth magnets 206 inserted in the one or more machine-washable layers of cloth 204 and the number of adhesive magnets 208 secured to human face 102.

Cloth magnets 206 and adhesive magnets 208 are safe to make contact with human face 102 and/or use in close proximity to human face 102. Cloth magnets 206 and adhesive magnets 208 may include similar or the same magnets as those used in magnetic jewelry, magnetic bracelets, magnetic earrings, among other types of commonly used magnets. Cloth magnets 206 and adhesive magnets 208 may include ferromagnetic metals that are strongly attracted by magnetic force, such as those described herein. Cloth magnets 206 and adhesive magnets 208 may include ferromagnetic metals such as iron, nickel, cobalt, gadolinium, dysprosium and alloys such as steel that also contain specific ferromagnetic metals. Cloth magnets 206 and adhesive magnets 208 may include ferromagnetic metals commonly used to make permanent magnets. Cloth magnets 206 and adhesive magnets 208 may also include smaller and yet powerful magnets, such as neodymium, that are implantable beneath human skin. In some instance, adhesive magnets 208 may be implanted underneath human skin and the number of cloth magnets 206 may secure the one or more machine-washable layers of cloth 204 over the human face 102 based at least on a magnetic attraction or force between the number of cloth magnets 206 and the number of implanted magnets 208 underneath the human skin of human face 102.

The number of adhesive magnets 208 may include a number of dual-sided adhesives 210, where a number of dual-sided adhesives 210 may be configured to secure the number of adhesive magnets 208 to human face 102. In some instances, a number of dual-sided adhesives 210 secures, attaches, and/or positions the number of adhesive magnets 208 to human face 102.

A number of cloth magnets 206 may be configured to secure the one or more machine-washable layers of cloth 204 over human face 102 based at least on a magnetic attraction and/or force between the number of cloth magnets 206 inserted in one or more machine-washable layers of cloth 204 and the number of adhesive magnets 210 secured to human face 102. One or more machine-washable layers of cloth 204 covers nose 104 and mouth 106 of human face 102 of human person 100 without one or more machine-washable layers of cloth 204 making contact with one or more ears 112 of human person 100, neck 108 of human person 100, a back of head 110 human person 100, and a top of head 110 of human person 100.

A number of dual-sided adhesives 210 may be removable or removed from the number of adhesive magnets 208 and replaceable or replaced with a second number of dual-sided adhesives 210 configured to secure, attach, and/or position the number of adhesive magnets 208 to human face 102. In some instances, a number of cloth magnets 206 may be inserted between a first layer (outer layer) and a second layer (inner layer) of the one or more machine-washable layers of cloth 204. For example, cloth magnets 206 may be inserted and stitched and/or sewed between the first layer (outer layer) and the second layer (inner layer) of the one or more machine-washable layers of cloth 204. A number of cloth magnets 206 in between the first layer and the second layer may secure the one or more machine-washable layers of cloth 204 over human face 102 based at least on the strength of the magnetic force or attraction through the second layer to the number of adhesive magnets 208 secured to human face 102.

FIG. 2D illustrates a drawing of an inside view of face covering 204, in accordance with example embodiments. As shown, one or more machine-washable layers of cloth of face covering 204 includes a number of pockets 212 and a number of insertion slots 214. A number of cloth magnets 206 may be insertable in a number of insertion slots 214 to secure and/or hold the number of cloth magnets 206. A number of cloth magnets 206 may secure one or more machine-washable layers of cloth of the face covering 204 over human face 102 based at least on the magnetic force and/or attraction of a number of cloth magnets 206 through the number of insertion slots 214 to a number of adhesive magnets 208 secured to human face 102.

In some embodiments, a lightweight metallic wire may be insertable in and removable from the one or more machine-washable layers of cloth of face covering 204. For example, the lightweight metallic wire may be inserted and/or sewn in along the edges of one or more machine-washable layers of cloth of the face covering 204. The lightweight metallic wire may be bendable, flexible, and/or adaptable to meet one or more contours of human face 102, such as facial areas 109, including the cheek bones. The lightweight metallic wire may be configured to secure one or more machine-washable layers of cloth of face covering 204 over human face 102 based at least on a magnetic attraction and/or force between the lightweight metallic wire inserted in the one or more machine-washable layers of cloth of the face covering 204 and a number of adhesive magnets 208 secured to human face 102.

In some embodiments, one or more machine-washable layers of cloth of the face covering 204 may include a single layer of cloth. Further, a second face covering that takes the form of face covering 204 may include a double layer of cloth and a third face covering that also takes the form of face covering 204 may include a triple layer of cloth. In such instance, face covering 204 with the single layer of cloth may be removable from a number of adhesive magnets 208 secured to human face 102 and replaceable with at least one of the second face covering and the third face covering without removing a number of adhesive magnets 208 secured to human face 102. As such, the first face covering 204, the second face covering, and the third face covering may be interchangeable. The first face covering 204, the second face covering, and the third face covering may also be stacked and secured to human face 102 for added protection, among other possibilities. Notably, the single layer of cloth may be removable and replaceable with the second face covering and/or the third face covering without making contact with one or more ears 112 of the person 100, neck 108 of human person 100, a back of head 110 of the human person 100, and a top portion of head 110 of human person 100.

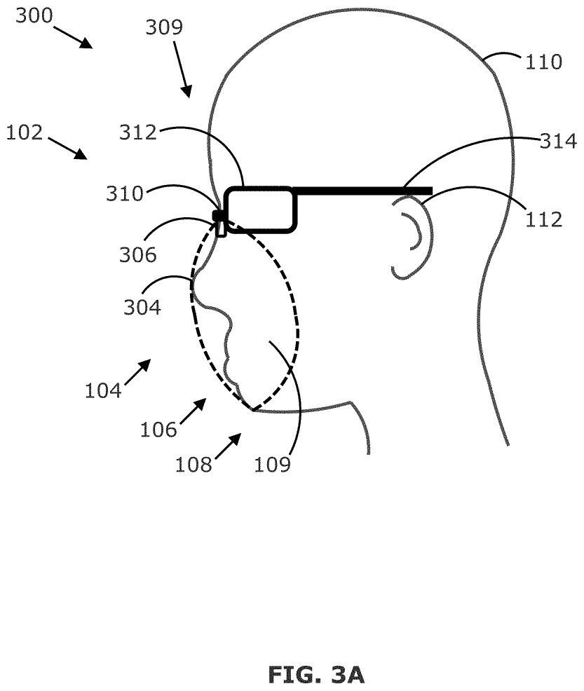

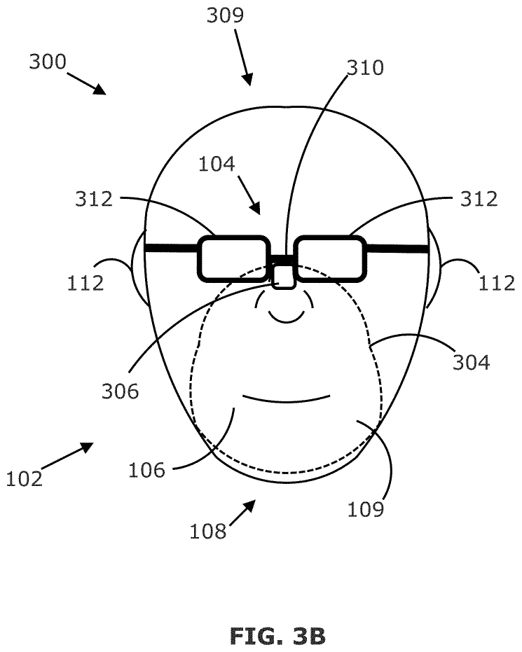

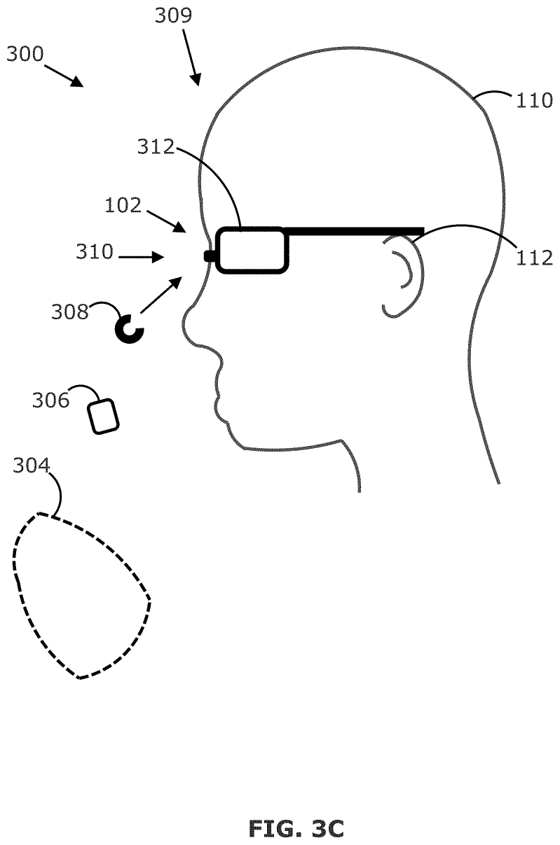

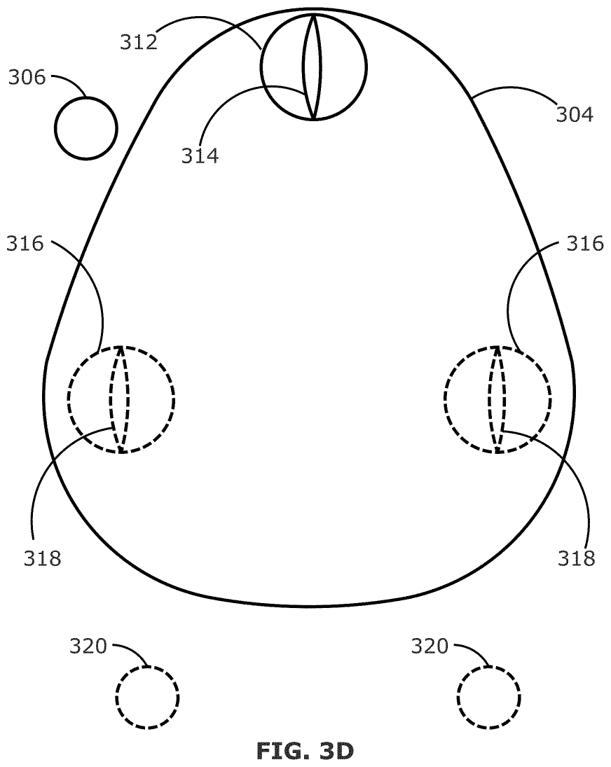

FIG. 3A illustrates a drawing of face covering 304 in relation to a pair of eyeglasses 309, in accordance with example embodiments. As shown, human person 300 may have one or more similarities with human person 100 described above. For example, human person 300 may have human face 102, including nose 104, mouth 106, chin and/or neck 108, possibly as described above. Further, human person 300 may include head 110, ears 112, among various other parts of the body of human person 300, possibly as described above for human person 100. Yet, human person 300 may have a number of different characteristics in relation to the body parts described above. Pair of eyeglasses 309, also referred to herein as eyeglasses 309, may include bridge 310, frames 312, and supports 314, among other components and/or parts of eyeglasses and/or eyewear known in the arts. As shown, bridge 310 may rest on nose or nose bridge 104 and supports 314 may rest over ears 112. One or more cloth magnets 306 may connect, attach, and/or secure with bridge 310, thereby securing one or more machine-washable layers of cloth of face covering 304 to eyeglasses 309. Eyeglasses 309 may take various forms including full frame glasses, semi-rimless glasses, and/or rimless glasses, among various other types of glasses with variable structures.

Contemplating adhesive magnets 208 described above, at least one of a number of adhesive magnets 208 may be configured to attach to eyeglasses 309. Further, at least one of a number of cloth magnets 306 may be configured to secure one or more machine-washable layers of cloth 304 over human face 102 based at least on a magnetic attraction and/or force between at least one of the number of cloth magnets 306 inserted, embedded, and/or attached to the one or more machine-washable layers of cloth 304 and at least one of a number of adhesive magnets 208 secured to, attached to, and/or embedded in eyeglasses 309. As such, one or more machine-washable layers of cloth 304 may cover nose 104 and mouth 106 of human face 102 of human person 300 without one or more machine-washable layers of cloth 304 making contact with one or more ears 112 of human person 300, neck 108 of human person 300, a back of head 110 of human person 300, and a top of head 110 of human person 300, among other parts of the body of human person 300.

FIG. 3B illustrates a drawing of face covering 304 in relation to a pair of glasses 309 from a frontal view, in accordance with example embodiments. As shown, human person 300 may include nose 104, mouth 106, chin and/or neck 108, facial areas 109 adjacent to nose 104 and/or mouth 106, and/or ear 112, as described above. Further, glasses 309 may include bridge 310, frames 312, and/or supports 314, as described above.

As shown, at least one of a number of cloth magnets 306 may be configured to secure one or more machine-washable layers of cloth 304 over human face 102 based at least on a magnetic attraction and/or force between at least one of a number of cloth magnets 306 inserted, embedded, and/or attached to the one or more machine-washable layers of cloth 304 and the at least one of a number of adhesive magnets 208 secured to, attached to, and/or embedded in eyeglasses 309. As such, one or more machine-washable layers of cloth 304 may covers nose 104 and mouth 106 of human face 102 of human person 300 without one or more machine-washable layers of cloth 304 making contact with one or more eyes 111, one or more ears 112 of human person 300, a neck of human person 300, a back of head 110 of human person 300, and a top of head 110 of the human person 300, among other parts of the body of human person 300.

It should be noted that one or more machine-washable layers of cloth 304 may be flexible and adaptable to fit to the contours of human face 102 of human person 300 and/or human face 102 of human person 100, among various other human faces of human persons. For example, cloth 304 may be adaptable to fit underneath frames 312, which may prevent fogging up frames or lenses 312. In particular, one or more machine-washable layers of cloth 304 may fit the contours of nose 104 and facial areas 109 around nose 104 to prevent heated air from human person 300's breath moving upwards to frames 312.

FIG. 3C illustrates a drawing of an exploded view of face covering 304 in relation to pair of glasses 309, in accordance with example embodiments. As shown, human person 300 includes face 102, head 110, and ears 112, as described above. Further, glasses 309 include bridge 310 and frames 312, as described above. Yet further, one or more machine-washable layers of cloth 304, at least one of the number of cloth magnets 306, and magnet 308 are shown to secure the one or more machine-washable layers of cloth 304 to cover human face 102. At least one of a number of magnets 308 may be adjustable to attach to bridge 310 of eyeglasses 309. For example, magnets 308 may be bendable (e.g., U-shaped) to wrap around bridge 308 and/or possibly clip on to bridge 308, among other possibilities.

FIG. 3D illustrates a drawing of an inside view of face covering 304, in accordance with example embodiments. As shown, one or more machine-washable layers of cloth 304 may include at least one pocket 312 and at least one insertion slot 314. Additional pockets 316 and insertion slots 318 illustrated with dashed lines may also be included, where pockets 316 and insertion slots 318 may take the form of pockets 212 and insertion slots 214, respectively, as described above. Further, a number of cloth magnets 320 may be insertable into pockets 318 similarly as described above with cloth magnets 206 insertable into pockets 214.

At least one of a number of cloth magnets 306 may be insertable or inserted in at least one insertion slot 314 to hold and/or secure at least one of the number of cloth magnets 306. At least one of a number of cloth magnets 306 may secure the one or more machine-washable layers of cloth 304 over human face 102 based on a magnetic force and/or attraction through the insertion slot 314 between the cloth magnet 306 with at least one of a number of adhesive magnets 308 attachable and/or attached to bridge 310 of eyeglasses 309.

In some embodiments, a lightweight metallic wire may be insertable in and removable from the one or more machine-washable layers of cloth of face covering 304. For example, the lightweight metallic wire may be inserted and/or sewed in along the edges of one or more machine-washable layers of cloth of the face covering 304. The lightweight metallic wire may be bendable, flexible, and adaptable to meet one or more contours of human face 102, such as facial areas 109 including contours around the cheek bones. The lightweight metallic wire may be configured to secure one or more machine-washable layers of cloth of face covering 304 over human face 102 based at least on a magnetic attraction and/or force between the lightweight metallic wire inserted in the one or more machine-washable layers of cloth of the face covering 304 and the number of adhesive magnets 308 secured to eyeglasses 309.

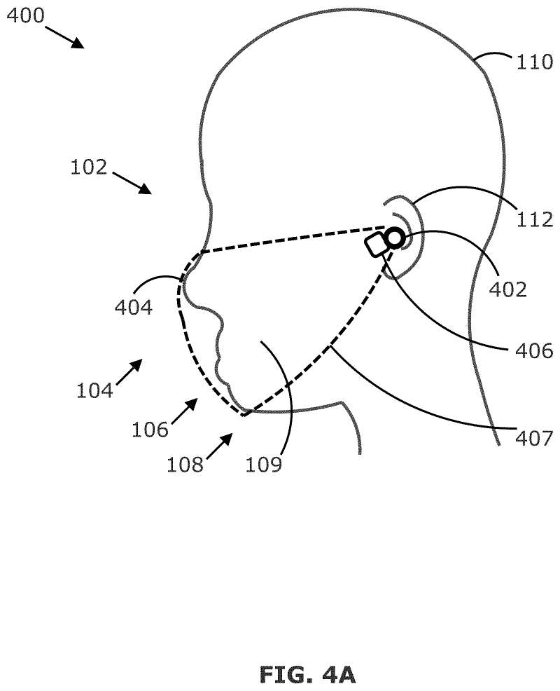

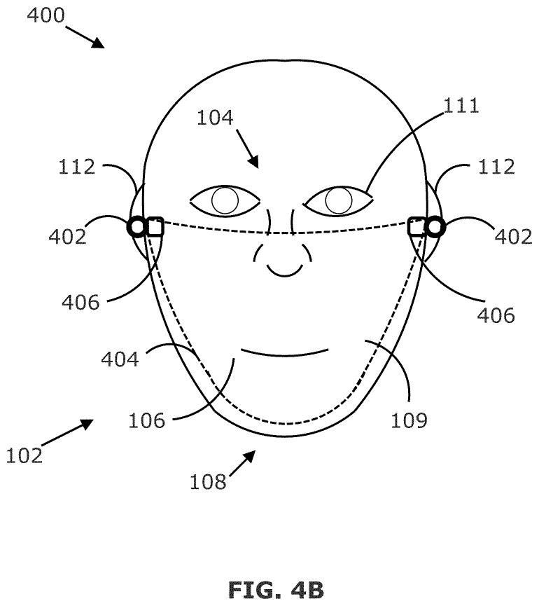

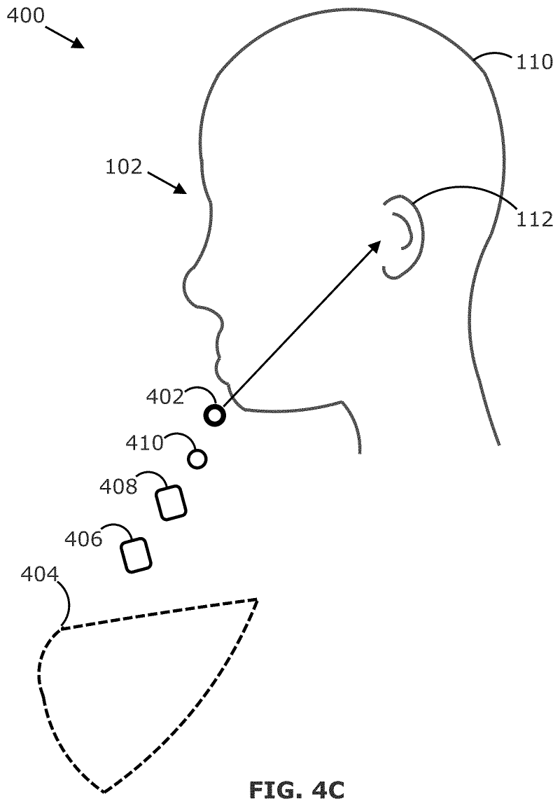

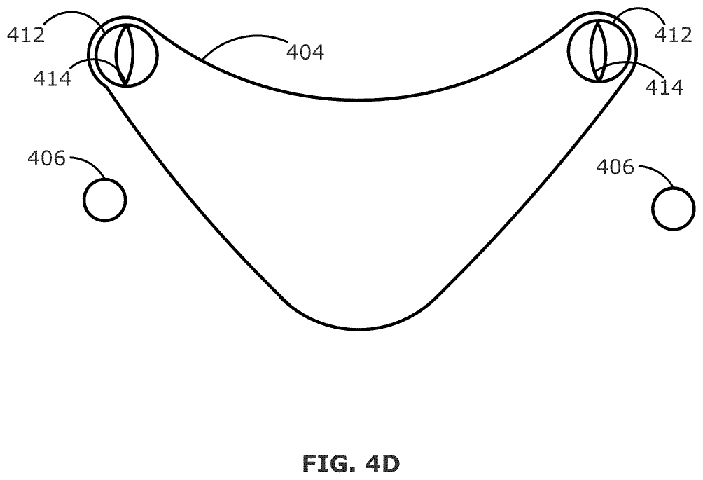

FIG. 4A illustrates a drawing of face covering 404 in relation to a pair of wireless headphones 402, in accordance with example embodiments. As shown, human person 400 may have one or more similarities with human persons 100 and/or 300 described above. For example, human person 400 may have human face 102, including nose 104, mouth 106, chin and/or neck 108, facial areas 109, possibly as described above. Further, human person 400 may include head 110, ears 112, among various other parts of the body of human person 400, possibly as described above for human persons 100 and 300. Yet, human person 400 may have different shapes and sizes of the body parts described above in comparison to human person 200 and 300.

Contemplating adhesive magnets 308 described above, a number of adhesive magnets 308 may be configured to attach to pair of wireless headphones 402, also referred to herein as wireless headphones 402. A number of cloth magnets 406 may be configured to secure one or more machine-washable layers of cloth of face coving 404 over human face 102 based at least on a magnetic force or attraction between the number of cloth magnets 406 inserted in, embedded in, and/or attached to one or more machine-washable layers of cloth of face covering 404 and a number of adhesive magnets 308 secured, attached, and/or clipped on to the pair of wireless headphones 402. As such, one or more machine-washable layers of cloth 404 may cover nose 104 and mouth 106 of human face 102 of human person 400 without one or more machine-washable layers of cloth 404 making contact with one or more ears 112 of human person 400, neck 108 of human person 400, a back of head 110 of human person 400, and a top of head 110 of the human person 400, among other parts of the body of human person 400.

FIG. 4B illustrates a drawing of face covering 304 in relation to a pair of wireless headphones 402 from a frontal view, in accordance with example embodiments. As shown, human person 400 may include nose 104, mouth 106, chin and/or neck 108, facial areas 109 adjacent to nose 104 and/or mouth 106, eyes 111, and/or ears 112, as described above.

As shown, a number of cloth magnets 406 may be configured to secure one or more machine-washable layers of cloth 404 over human face 102 based at least on a magnetic attraction and/or force between a number of cloth magnets 406 inserted, embedded, and/or attached to the one or more machine-washable layers of cloth 404 and a number of adhesive magnets 308 secured to, attached to, and/or embedded in wireless headphones 402. As such, one or more machine-washable layers of cloth 404 may cover nose 104 and mouth 106 of human face 102 of human person 400 without one or more machine-washable layers of cloth 404 making contact with one or more eyes 111, one or more ears 112 of human person 400, a neck of human person 400, a back of head 110 of human person 400, and a top of head 110 of human person 400, among other parts of the body of human person 400.

FIG. 4C illustrates a drawing of an exploded view of face covering 404 in relation to a pair of wireless headphones 402, in accordance with example embodiments. As shown, human person 400 includes face 102, head 110, and ears 112, as described above. Yet further, one or more machine-washable layers of cloth 404, a number of cloth magnets 406, and magnet 408 are shown to secure the one or more machine-washable layers of cloth 404 to cover face 102. A number of magnets 408 may be adjustable to attach to wireless headphones. For example, magnets 408 may be bendable similarly to magnets 308 described above. As such, magnets 408 may be bendable to wrap around various portions of wireless headphones 402. Yet, a number of adhesive magnets 408 may include a number of dual-sided adhesives 410, where a number of dual-sided adhesives 410 may be configured to secure a number of the adhesive magnets 408 to wireless headphones 402. In some instances, wireless headphones 402 may include components configured to be placed in ears 112 and portions configured to be external to ears 112, such as stem-shaped components, tube-shaped components, and/or round-shaped components, among other possibilities. As such, a number of magnets 408 may be adjustable, flexible, and/or bendable to wrap around wireless headphones 404 to secure one or more machine-washable layers of cloth 404 to cover face 102, as described above.

FIG. 4D illustrates a drawing of an inside view of face covering 404, in accordance with example embodiments. As shown, one or more machine-washable layers of cloth 404 may include a number of pockets 412 and a number of slots 414. It may be contemplated that additional pockets 312 and 316, and insertion slots 314 and 318, respectively, may also be included, as provided in one or more descriptions above. Pockets 412 and insertion slots 414 may take the form of pockets 312, 316, and insertion slots 314, 318, respectively, as described above. Further, a number of cloth magnets 406 may be insertable into pockets 412 as described above with cloth magnets 306, 320 insertable into pockets 312, 316, respectively. A number of cloth magnets 406 may secure the one or more machine-washable layers of cloth 404 over human face 102 based at least on magnetic attraction between a number of cloth magnets 406 through a number of insertion slots 414 to a number of adhesive magnets 408 attached to wireless headphones 402.

As noted above, a lightweight metallic wire may be insertable in and removable from one or more machine-washable layers of cloth of face covering 404. For example, the lightweight metallic wire may be inserted and/or sewed in along the edges of one or more machine-washable layers of cloth of the face covering 404. The lightweight metallic wire may be adjustable and/or bendable to meet one or more contours of human face 102, including facial areas 109, such as contours of cheek bones of human face 102. The lightweight metallic wire may be configured to secure one or more machine-washable layers of cloth of face covering 404 over human face 102 based at least on a magnetic attraction and/or force between the lightweight metallic wire inserted in the one or more machine-washable layers of cloth of the face covering 404 and the number of adhesive magnets 408 attached to wireless headphones 402.

In some embodiments, human person 400 may be wearing wireless headphones 402 and glasses 309 described above. At least one of a number of adhesive magnets 308 may be attached to the pair of eyeglasses 309 and one or more other adhesive magnets 408 from a number of adhesive magnets may be attached to the pair of wireless headphones 402. One or more machine-washable layers of cloth of the face covering 404 may include pockets 312 and 412 where cloth magnets 306 and 406 may be insertable into pockets 312 and 412, respectively. As such, a number of cloth magnets 306 and 406 may be configured to secure the one or more machine-washable layers of cloth 404 over human face 102 based at least on one or more magnetic attractions between a number of cloth magnets 306 and 406 in the one or more machine-washable layers of cloth 404 and a number of adhesive magnets 308 attached to pair of eyeglasses 309 and one or more other adhesive magnets 408 attached to pair of wireless headphones 402. In some embodiments, portion 407 one or more machine-washable layers of cloth 404 may be folded or folded inside to take the form of one or more machine-washable layers of cloth of face covering 304 and/or 204, among other possibilities.

It should be noted that face coverings 204, 304, and 404 may take the form of face masks, possibly with one or more filters. For example, referring back to FIG. 2A-2D, face covering 204 may be a face mask with one or more filters. As such, one or more layers of cloth and/or one or more filters of face mask 204 may be used to cover human face 102. A number of magnets 206 may be insertable in and removable from one or more layers of cloth and/or one or more filters of face mask 204. A number of adhesive magnets 208 may include a number of dual-sided adhesives 210, where a number of dual-sided adhesives 210 may be configured to secure a number of the adhesive magnets 208 to human face 102. A number of cloth magnets 206 may be configured to secure the one or more layers of cloth and/or one or more filters of face mask 204 over the human face 102 based at least on a magnetic attraction between a number of cloth magnets 206 inserted in the one or more layers of cloth and/or one or more filters of face mask 204 and a number of adhesive magnets 208 secured to human face 102.

Face mask 204 may be a surgical mask and a respirator mask, and/or a ventilator mask, among other types of masks. In some embodiments, face mask 204 is a surgical mask, a second face mask 304 may take the form of a respirator mask (e.g., N95 respirator mask). As such, surgical mask 204 may be removable (removed) from a number of adhesive magnets 208 secured to human face 102 and replaceable (replaced) with respirator mask 304 without removing a number of adhesive magnets 208 secured to human face 102 of human person 100. Surgical mask 204 may be removed and replaced by respirator mask 304 (and vice versa) without making contact with one or more ears 112 of human person 100, neck 108 of human person 100, a back of head 110 of human person 100, and a top of head 110 of human person 100.

As noted, a lightweight metallic wire may be insertable in and removable from the one or more layers of cloth of face mask 204. For example, the lightweight metallic wire may be flexible, bendable, and adaptable to meet or make contact with one or more contours of the human face 102 including contours of facial areas 109, such as cheek bone structures. Further, the lightweight metallic wire may be configured to secure the one or more layers of cloth of face mask 204 over human face 102 based at least on a magnetic force and/or attraction between the lightweight metallic wire inserted in the one or more of layers cloth of face mask 204 and the plurality of adhesive magnets secured to human face 102.

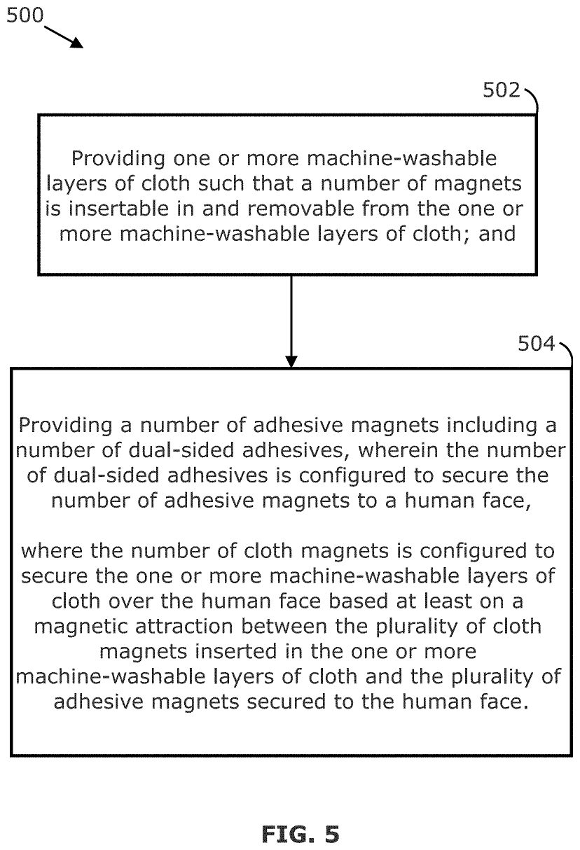

FIG. 5 illustrates a flowchart 500, in accordance with example embodiments. At step 502, flowchart 500 includes providing one or more machine-washable layers of cloth such that a number of magnets is insertable in and removable from the one or more machine-washable layers of cloth. As described above, one or more machine-washable layers of cloth of face coverings 204, 304, and 404 may be provided such that a number of magnets 206, 306, and 406 may be inserted in and removed from the one or more machine-washable layers of cloth of face coverings 204, 304, and 404, respectively.

In some embodiments, providing the one or more machine-washable layers of cloth of face coverings 204, 304, and 404 may include obtaining, making, and/or sewing together the one or more machine-washable layers of cloth and a number of the pockets 212, 312, 316, and/or 412, and insertion slots 214, 314, 318, and/or 414, respectively.

At step 504, flowchart 500 includes providing a number of adhesive magnets including a number of dual-sided adhesives, where the number of dual-sided adhesives is configured to secure the number of adhesive magnets to a human face, where the number of cloth magnets is configured to secure the one or more machine-washable layers of cloth over the human face based at least on a magnetic attraction between the number of cloth magnets inserted in the one or more machine-washable layers of cloth and the number of adhesive magnets secured to the human face. As described above, a number of adhesive magnets 208 may include a number of dual-sided adhesives 210, where the number of dual-sided adhesives 210 may be configured to secure the number of adhesive magnets 208 to human face 102, where the number of cloth magnets 206 may be configured to secure the one or more machine-washable layers of cloth of face covering 204 over human face 102 based at least on a magnetic attraction between the number of cloth magnets 206 inserted in the one or more machine-washable layers of cloth of face covering 204 and the number of adhesive magnets 208 secured to human face 102.

In some embodiments, the flowchart 500 may also include providing a number of pockets 212, 312, 316, and/or 412 and a number of insertion slots 214, 314, 318, and/or 414 with the one or more machine-washable layers of cloth of face covering 204, 304, and/or 404, respectively. For example, the number of pockets 212, 312, and/or 412 and the number of insertion slots 214, 314, and/or 404 may be sewed on the one or more machine-washable layers of cloth of face coverings 204, 304, and/or 404, respectively. As such, the number of cloth magnets 206, 306, and 406 may be insertable in the number of insertion slots insertion slots 214, 314, 318, and/or 414 to secure the number of cloth magnets 206, 306, and 406, respectively. As such, the number of cloth magnets 206, 306, and 406 may be provided to secure the one or more machine-washable layers of cloth of face coverings 204, 304, and 404 over human face 102 based at least on the magnetic attraction through the number of insertion slots 214, 314, and/or 414 to the number of adhesive magnets 208, 308, and/or 408, respectively.

Further, the flowchart 500 may include packaging and delivering the one or more machine-washable layers of cloth of face coverings 204, 304, and 404 with the number of cloth magnets 206, 306, and 406 insertable and removable from the one or more machine-washable layers of cloth of face coverings 204, 304, and 404, respectively. Further, the flowchart 500 may also include packaging and delivering the number of adhesive magnets 208, 308, and/or 408, and potentially the number of dual-sided adhesives 210.

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features of apparatus, methods, and devices with reference to the accompanying figures. The example embodiments described herein and in the figures are not meant to be limiting. It should be understood that other embodiments can include more or less of each element shown in a given figure. Some of the illustrated elements can be combined or omitted. Further, example embodiments can include elements that are not illustrated in the figures. Other embodiments can be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all the message flow diagrams, scenarios, and flow charts in the figures and discussed herein, each step, block can represent various types of product development, assembly, and manufacturing processes in accordance with example embodiments. Alternative embodiments, for example, operations described as steps, blocks, and processes can be executed out of order from that shown or discussed, including substantially concurrently or in reverse order, depending on the functionality involved. Further, more or few blocks and/or processes can be used with any of the flow charts discussed herein.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

What is claimed is:

1. A face covering apparatus comprising:
one or more machine-washable layers of cloth, wherein a plurality of magnets is insertable in and removable from the one or more machine-washable layers of cloth;
a plurality of adhesive magnets comprising a plurality of dual-sided adhesives, wherein the plurality of dual-sided adhesives is configured to secure the plurality of adhesive magnets to a human face,
wherein the plurality of cloth magnets is configured to secure the one or more machine-washable layers of cloth over the human face based at least on a magnetic attraction between the plurality of cloth magnets inserted in the one or more machine-washable layers of cloth and the plurality of adhesive magnets secured to the human face.

2. The face covering apparatus of claim 1, wherein the one or more machine-washable layers of cloth covers a nose and a mouth of the human face of a human person without the one or more machine-washable layers of cloth making contact with one or more ears of the human person, a neck of the human person, a back of a head of the human person, and a top of the head of the human person.

3. The face covering apparatus of claim 1, wherein the plurality of dual-sided adhesives is removable from the plurality of adhesive magnets and replaceable with a second plurality of dual-sided adhesives configured to secure the plurality of adhesive magnets to the human face.

4. The face covering apparatus of claim 1, wherein the plurality of cloth magnets is inserted between a first layer and a second layer of the one or more machine-washable layers of cloth,
wherein the plurality of cloth magnets secure the one or more machine-washable layers of cloth over the human face based at least on the magnetic attraction through the second layer to the plurality of adhesive magnets secured to the human face.

5. The face covering apparatus of claim 1, wherein the one or more machine-washable layers of cloth comprises a plurality of pockets and a plurality of insertion slots,
wherein the plurality of cloth magnets is insertable in the plurality of insertion slots to secure the plurality of cloth magnets, and
wherein the plurality of cloth magnets secure the one or more machine-washable layers of cloth over the human face based at least on the magnetic attraction through the plurality of insertion slots to the plurality of adhesive magnets secured to the human face.

6. The face covering apparatus of claim 1, wherein a lightweight metallic wire is insertable in and removable from the one or more machine-washable layers of cloth, and wherein the lightweight metallic wire is bendable to meet one or more contours of the human face,
wherein the lightweight metallic wire is configured to secure the one or more machine-washable layers of cloth over the human face based at least on a magnetic attraction between the lightweight metallic wire inserted in the one or more machine-washable layers of cloth and the plurality of adhesive magnets secured to the human face.

7. The face covering apparatus of claim 1, wherein at least one of the plurality of adhesive magnets is configured to attach to a pair of eyeglasses,
wherein at least one of the plurality of cloth magnets is configured to secure the one or more machine-washable layers of cloth over the human face based at least on a second magnetic attraction between the at least one of the plurality of cloth magnets in the one or more machine-washable layers of cloth and the at least one of the plurality of adhesive magnets secured to the pair of eyeglasses, and
wherein the one or more machine-washable layers of cloth covers a nose and a mouth of the human face of a human person without the one or more machine-washable layers of cloth making contact with one or more ears of the human person, a neck of the human person, a back of a head of the human person, and a top of the head of the human person.

8. The face covering apparatus of claim 7, wherein the at least one of the plurality of adhesive magnets is adjustable to attach to a bridge of the pair of eyeglasses,
wherein the one or more machine-washable layers of cloth comprises at least one pocket and at least one insertion slot,
wherein the at least one of the plurality of cloth magnets is insertable in the at least one insertion slot to secure the at least one of the plurality of cloth magnets with the one or more machine-washable layers of cloth, and
wherein the at least one of the plurality of cloth magnets secures the one or more machine-washable layers of cloth over the human face based at least on the second magnetic attraction through the at least one insertion slot to the at least one of the plurality of adhesive magnets attached to the bridge of the pair of eyeglasses.

9. The face covering apparatus of claim 1, wherein the plurality of adhesive magnets is configured to attach to a pair of wireless headphones,
wherein the plurality of cloth magnets is configured to secure the one or more machine-washable layers of cloth over the human face based at least on a second magnetic attraction between the plurality of cloth magnets in the one or more machine-washable layers of cloth and the plurality of adhesive magnets secured to the pair of wireless headphones, and
wherein the one or more machine-washable layers of cloth covers a nose and a mouth of the human face of a human person without the one or more machine-washable layers of cloth making contact with a neck of the human person, a back of a head of the human person, and a top of the head of the human person.

10. The face covering apparatus of claim 9, wherein the plurality of adhesive magnets is adjustable to attach to the pair of wireless headphones,
wherein the one or more machine-washable layers of cloth comprises a plurality of pockets and a plurality of insertion slots,
wherein the plurality of cloth magnets is insertable in the plurality of insertion slots to secure the plurality of cloth magnets, and
wherein the plurality of cloth magnets secures the one or more machine-washable layers of cloth over the human face based at least on the second magnetic attraction through the plurality of insertion slots to the at least one of the plurality of adhesive magnets attached to the pair of wireless headphones.

11. The face covering apparatus of claim 1, wherein at least one of the plurality of adhesive magnets is attached to a pair of eyeglasses, and wherein one or more other adhesive magnets from the plurality of adhesive magnets is attached to a pair of wireless headphones, and
wherein the plurality of cloth magnets is configured to secure the one or more machine-washable layers of cloth over the human face based at least on one or more magnetic attractions between the plurality of cloth magnets in the one or more machine-washable layers of cloth and the at least one of the plurality of adhesive magnets attached to the pair of eyeglasses and the one or more other adhesive magnets attached to the pair of wireless headphones.

12. The face covering apparatus of claim 1, wherein the one or more machine-washable layers of cloth comprises a single layer of cloth, and wherein a second face covering comprises a double layer of cloth, and wherein a third face covering comprises triple layer of cloth, and
wherein the single layer of cloth is removable from the plurality of adhesive magnets secured to the human face and replaceable with at least one of the second face covering and the third face covering without removing the plurality of adhesive magnets secured to the human face, and
wherein the single layer of cloth is removable and replaceable without making contact with one or more ears of the person, a neck of the human person, a back of a head of the human person, and a top of the head of the human person.

13. The face covering apparatus of claim 1, wherein the plurality of dual-sided adhesives comprises at least one of the following organic adhesives comprising starch adhesives, paste-based adhesives, natural resin adhesives, and animal adhesives.

14. A face mask apparatus comprising:
one or more layers of cloth, wherein a plurality of magnets is insertable in and removable from the one or more layers of cloth;
a plurality of adhesive magnets comprising a plurality of dual-sided adhesives, wherein the plurality of dual-sided adhesives is configured to secure the plurality of adhesive magnets to a human face,
wherein the plurality of cloth magnets is configured to secure the one or more layers of cloth over the human face based at least on a magnetic attraction between the plurality of cloth magnets inserted in the one or more layers of cloth and the plurality of adhesive magnets secured to the human face.

15. The face mask apparatus of claim 14, wherein the face mask is at least one of a surgical mask and a respirator mask.

16. The face mask apparatus of claim 14, wherein the face mask is a surgical mask and a second face mask is a respirator mask,
wherein the surgical mask is removable from the plurality of adhesive magnets secured to the human face and replaceable with the respirator mask without removing the plurality of adhesive magnets secured to the human face of a human person, and
wherein the surgical mask is removable and replaceable without making contact with one or more ears of the human person, a neck of the human person, a back of a head of the human person, and a top of the head of the human person.

17. The face mask apparatus of claim 14, wherein a lightweight metallic wire is insertable in and removable from the one or more layers of cloth, and wherein the lightweight metallic wire is bendable to meet one or more contours of the human face,
wherein the lightweight metallic wire is configured to secure the one or more layers of cloth over the human face based at least on a second magnetic attraction between the lightweight metallic wire inserted in the one or more of layers of cloth and the plurality of adhesive magnets secured to the human face.

18. A method comprising:
providing one or more machine-washable layers of cloth such that a plurality of magnets is insertable in and removable from the one or more machine-washable layers of cloth; and
providing a plurality of adhesive magnets comprising a plurality of dual-sided adhesives, wherein the plurality of dual-sided adhesives is configured to secure the plurality of adhesive magnets to a human face,
wherein the plurality of cloth magnets is configured to secure the one or more machine-washable layers of cloth over the human face based at least on a magnetic attraction between the plurality of cloth magnets inserted in the one or more machine-washable layers of cloth and the plurality of adhesive magnets secured to the human face.

19. The method of claim 18, further comprising providing a plurality of pockets and a plurality of insertion slots with the one or more machine-washable layers of cloth,
wherein the plurality of cloth magnets is insertable in the plurality of insertion slots to secure the plurality of cloth magnets, and
wherein the plurality of cloth magnets secure the one or more machine-washable layers of cloth over the human face based at least on the magnetic attraction through the plurality of insertion slots to the plurality of adhesive magnets secured to the human face.

20. The method of claim 18, further comprising packaging and delivering the one or more machine-washable layers of cloth with the plurality of cloth magnets insertable and removable from the one or more machine-washable layers of cloth.
